SEQUENCE LISTING (4 sequences)

SEQ ID NO: 1 - DNA, 1518 bp, Artificial Sequence
  fragment of L1 gene
atgtctctttggctgcctagtgaggccactgtctacttgcctcctgtcccagtatctaaggttgtaagcacggatgaatatgttgcacgcacaaacatatattatcatgcagggacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggccgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacggagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 2 - DNA, 1452 bp, Artificial Sequence
  fragment of L1 gene
atgtctctttggctgcctagtgaggccactgtctacttgcctcctgtcccagtatctaaggttgtaagcacggatgaatatgttgcacgcacaaacatatattatcatgcagggacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggccgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacggagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggataa

SEQ ID NO: 3 - DNA, 1518 bp, Artificial Sequence
  fragment of L1 gene
atgtctctttggctgcctagtgaggccactgtctacttgcctcctgtcccagtatctaaggttgtaagcacggatgaatatgttgcacgcacaaacatatattatcatgcagggacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggccgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacggagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 4 - DNA, 1452 bp, Artificial Sequence
  fragment of L1 gene
atgtctctttggctgcctagtgaggccactgtctacttgcctcctgtcccagtatctaaggttgtaagcacggatgaatatgttgcacgcacaaacatatattatcatgcagggacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggccgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacggagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcactttcctttaggacgcaaatttttactacaagcaggattgaaggccaaaccaaaatttacattaggataa

(19) United States
(12) Patent Application Publication
(10) Pub. No.: US 2005/0090435 A1
(43) Pub. Date: Apr. 28, 2005

(54) Pharmaceutical compositions, and a method of preparing and isolating said pharmaceutical compositions, and use of said pharmaceutical compositions for prophylactic treatment of lesions and carcinomas

(76) Inventors: Edward Peter Rybicki, Cape Town (ZA); Anna-Lise Williamson, Cape Town (ZA); Arvind Devshi Varsani, Cape Town (ZA)

Correspondence Address: BURNS DOANE SWECKER & MATHIS L L P, POST OFFICE BOX 1404, ALEXANDRIA, VA, 22313-1404, US

(21) Appl. No.: 10/487,719

(22) PCT Filed: Aug. 30, 2002
(86) PCT No.: PCT/IB02/03531

(51) Int. Cl. C12Q 1/68; C07H 21/04; A61K 38/17

(52) U.S. Cl. 514/12; 530/350; 435/69.1; 435/320.1; 435/325; 435/6; 536/23.72

(57) ABSTRACT

This invention provides isolated nucleotide sequences disclosed in the specification, amino acid sequences deduced from the nucleotide sequences, constructs and/or vectors comprising and or expressing the nucleotide sequences, and there use in transforming plant cells to produce proteins or fragments of proteins. These proteins or fragments can be used in the manufacture of pharmaceutical components that can be used in a prophylactic treatment against causative agents of lesions and carcinomas, in particular cervical lesions and carcinomas.

### BACKGROUND OF THE INVENTION

The invention relates to the use of papillomavirus proteins for preparing pharmaceutical compositions intended to generate an immunogenic response against papillomaviruses. The invention further comprises the use of the compositions for prophylactic treatment of lesions and carcinomas associated with papillomavirus infection. The invention further comprises diagnostic kits for use in the diagnosis of papillomavirus infection.

In this specification the following terms, phrases and/or clauses are to be understood to mean:

Construct—as used herein is synonymous with terms such as “conjugate”, “cassette”, and “hybrid” and includes the nucleotide sequence according to the invention, or parts thereof, which may be directly or indirectly linked to a promoter. The construct may contain or express a marker, which allows for the selection for the construct in a host cell.

“Expression vector”—as used herein means a construct capable of in vivo or in vitro expression.

“Expression” is understood to mean the production of a protein from a DNA template via transcription and translation.

“Fragment of the papillomavirus protein”—as used herein is intended to denote, in particular, any fragment of an amino acid sequence included in the amino acid of a papillomavirus protein which is capable of generating an immunogenic response directed against a papillomavirus, said fragment comprising at least 5 amino acids, preferably at least 10 amino acids and, more preferably at least 15 amino acids.

“Homologue”—as used herein means an entity having a certain homology with amino acid sequences or nucleotide sequences.

“Hybridisation”—as used herein encompasses the use of nucleotide sequences that are capable of hybridising to a nucleotide sequence of the present invention, or any fragment, variant, derivative, or homologue thereof.

“In silico”—parts of assays and/or processes described herein may be performed by use of suitable computational software. Such assays and/or processes so performed are to be understood to be encompassed herein.

“Nucleotide sequence”—as used herein is synonymous with the term “nucleotide acid sequence” and/or the term “polynucleic acid” and/or the term “polynucleotide” and includes genomic DNA, cDNA, recombinant DNA, synthetic DNA, and RNA, and any combinations of the aforementioned, also the nucleotide sequence may be double-stranded or single-stranded whether representing the sense or the antisense strand. Preferably, the term “nucleotide sequence” means DNA.

“Operably linked”—as used herein refers to a juxtaposition wherein the promoter which is “operably linked” to a coding sequence is ligated in such manner that expression of the coding sequence is achieved under conditions compatible with the control sequences (i.e. inter alia promoters, enhancers and other expression regulation signals).

“Promoter”—as used herein is a nucleotide sequence that directs the transcription of a nucleotide sequence.

“Protein”—as used herein is intended to denote both peptides and polypeptides.

“TMV”—as used herein indicates Tobacco Mosaic Virus.

“Vector”—as used herein includes expression vectors, replicable vectors, transformation vectors, shuttle vectors, cosmids, plasmids, phages, viruses and yeast artificial chromosomes or any combination thereof.

“VLP”—as used herein indicates virus-like particles.

Papillomaviruses (PVs) belong to the taxonomic family Papillomaviridae, which are small, non-enveloped, double-stranded (ds) DNA viruses. These viruses infect a wide range of higher vertebrates and are highly species-specific. Upon infection, human papillomaviruses (HPVs) have a particular tropism for undifferentiated squamous epithelial cells and apart from being associated with plantar warts, flat warts, and genital warts (condyloma acuminata), HPV infections are also linked to a disease called epidermodysplasia verruciformis, a rare lifelong disease characterized by disseminating papillomas. There has, furthermore, been an epidemiological and biochemical link of certain papillomavirus isolates with urogenital cancers, including cervical, vulvar, penile cancer, and malignant transformation of epidermodysplasia verruciformis lesions. Of the characterized HPV types, about 27 have been identified as the causal agents of anogenital infections. HPV types 6, 11, 16, 18, 31, 33, 35 and 42 have been identified as being the most prevalent, whereas types 16, 18, 31, 33, 51 and 54 have been associated with anogenital carcinomas, believed to be of high risk, more particularly HPV-16 has been found in 95% of cervical cancers.

Until recently it was believed that high-risk genital HPVs were only transmitted during sexual intercourse, however studies indicate that infants acquire high-risk HPV infections from their mothers at birth. In addition to non-sexual parent-to-child transmission, investigators detected HPV DNA in vaginal swabs from women, in cervical-vaginal specimens from young girls, and in vulval swabs from 9 out of 61 women who claimed no history of sexual contact.

Initial HPV infection causes cervical intraepithelial neoplasia (CIN), more commonly known as pre-cancerous lesions. It has been found that in some women there is a spontaneous regression of CIN, but this is not always the case and lesions can persist for years, increasing the likelihood of progression towards cervical cancer.

Treatment methods ranging from cryotherapy, surgical excision and topical treatments do not often alleviate problems and for patients who do respond to the treatment, recurrence is often a problem. Ultimate destruction of all infected tissue does not appear to be feasible, since multifocal disease and latency are common features of the infections. Antiviral drugs designed to target and arrest viral replication seem to be ineffective in the case of HPV infections, firstly because the virus does not replicate in the cells that maintain the infection and secondly due to the fact that viral replication genes seem to get lost through integration, rearrangement and deletions.

Traditionally most prophylactic vaccines have consisted of live, attenuated virus or formalin-inactivated virus. Due to the difficulties and risks involved in generating large quantities of these traditional vaccines there has been great emphasis on developing a viral protein subunit vaccine.

### SUMMARY OF THE INVENTION

In accordance with the invention there are provided isolated nucleotide sequences set out in any one of FIGS. 4 to 7 (SEQ ID NO 1-4), or parts thereof, more particularly an isolated recombinant nucleotide sequence containing a papillomavirus coding sequence, still more preferably the isolated recombinant nucleotide sequence expresses a papillomavirus capsid protein, even more preferably the isolated nucleotide sequence is a plant/viral chimeric nucleotide sequence, most preferably the plant/viral chimeric nucleotide sequence is a human papillomavirus/plant chimeric sequence encoding an isolated nucleotide sequence selected from the group consisting of:
(i) papillomavirus L1 capsid protein;
(ii) a truncated papillomapapillomavirus L1 protein coding sequence which has been altered to include an in-frame C-terminal fusion peptide, preferably of a length determined not to interfere with self-assembly of the papillomapapillomavirus L1 protein into pentamers or larger aggregates; most preferably wherein the in-frame C-terminal fusion peptide sequence is derived from the L2 capsid protein, still more preferably wherein the in-frame C-terminal fusion peptide sequence is derived from the group consisting of:
   a. papillomavirus E2 protein;
   b. papillomavirus E7 protein;
   c. papillomavirus E6 protein;
(iii) papillomavirus L2 capsid protein; preferably wherein the L2 protein coding sequence has been altered to include an in-frame C-terminal fusion peptide selected from the group consisting of:
   a. papillomavirus E6 protein;
   b. a derivative of papillomavirus E6 protein;
   c. papillomavirus E7 protein;
   d. a derivative of papillomavirus E7 protein;
   e. papillomavirus E2 protein;
   f. a derivative of papillomavirus E2 protein
(iv) a papillomavirus non-structural protein selected from the group consisting of:
   a. E2 non-structural protein or part thereof;
   b. E6 non-structural protein or part thereof;
   c. E7 non-structural protein or part thereof,
(v) a papillomavirus L1 capsid protein modified as follows:
   a. HPV 11 OR HPV 16 L1 gene lacking nuclear localisation signal (NLS-, Δ483)
   b. HPV 11 OR HPV 16 L1 gene lacking 10 N-terminal codons (ΔN10)
   c. HPV 11 OR HPV 16 L1 gene lacking 10 N-terminal codons and NLS (ΔN10Δ483)
   d. HPV 11 OR HPV 16 L1 gene with a C to G mutation (pen) at aa428 (pen504)
   e. HPV 11 or HPV 16 L1 genes with pen and ΔN10 modifications
   f. HPV 11 or HPV 16 L1 genes with pen and Δ483 modifications
   g. HPV 11 or HPV 16 L1 genes with pen and ΔN10Δ483 modifications
(vi) any combination of (i) to (v).

Furthermore, there are provided deduced amino acid sequences of said isolated nucleotide sequences or parts thereof, more preferably the deduced amino acid sequence reflects preferred plant codon usage.

Furthermore, in accordance with the invention there are provided homologues, variants, and/or derivatives of the isolated nucleotide sequences, or parts thereof.

Still furthermore, in accordance with the invention there are provided complements of the isolated nucleotide sequences, or parts thereof.

Still furthermore, in accordance with the invention there are provided complements of the homologues, variants and derivatives of the isolated nucleotide sequences, or parts thereof.

Still furthermore, in accordance with the invention there are provided construct(s) and/or vector(s) comprising and/or expressing:
a. the isolated nucleotide sequences set out in any one of FIGS. 4 to 7, or parts thereof; or
b. homologues, variants or derivatives of the isolated nucleotide sequences, or parts thereof; or
c. complements of the isolated nucleotide sequences, or parts thereof; or
d. complements of the homologues, variants or derivatives of the isolated nucleotide sequences, or parts thereof; or
e. a nucleotide sequence that is capable of hybridising to:
   (i) the isolated nucleotide sequences, or parts thereof; or
   (ii) the homologues, variants or derivatives of the isolated nucleotide sequences, or parts thereof; or
   (iii) the complements of the isolated nucleotide sequences, or parts thereof; or
   (iv) the complements of the homologues, variants or derivatives of the isolated nucleotide sequences, or parts thereof; or
   (v) any combination of (e)(i)-(e)(iv);
f. any combination of (a)-(e),
   and a promoter, wherein the promoter is operably linked to the sequence of any one of (a)-(f).

Furthermore, there is provided a method of producing a papillomavirus protein, or parts thereof, comprising the steps of:
1) contacting a plant cell with a vector and/or construct as claimed herein;
2) cultivating the plant cell under conditions suitable for the production of a protein or fragment of a protein of:
   (a) the isolated nucleotide sequences set out in any one of FIGS. 4 to 7, or parts thereof; or
   (b) homologues, variants or derivatives of the isolated nucleotide sequences, or parts thereof; or
   (c) complements of the isolated nucleotide sequences, or parts thereof; or
   (d) complements of the homologues, variants or derivatives of the isolated nucleotide sequences, or parts thereof; or
   (e) a nucleotide sequence that is capable of hybridising to:
      i. the isolated nucleotide sequences, or parts thereof; or
      ii. the homologues, variants or derivatives of the isolated nucleotide sequences, or parts thereof; or
      iii. the complements of the isolated nucleotide sequences, or parts thereof; or
      iv. the complements of the homologues, variants or derivatives of the isolated nucleotide sequences, or parts thereof; or
      v. any combination of (e)(i)-(e)(iv);
   (f) any combination of (a)-(e),
      wherein, preferably, the plant cell is a Nicotiana benthamiana plant cell and, preferably, the conditions permit the spontaneous assembly of the proteins or fragments of proteins into immunogenic-response eliciting virus-like particles.

Furthermore, there is provided a method of isolating and purifying proteins or fragments of the proteins of the isolated nucleotide sequences set out in any one of FIGS. 4 to 7, or parts thereof.

Furthermore, in accordance with the invention there is provided a use of a papillomavirus protein, or fragment of a papillomavirus protein, which comprises:
a. the amino acid sequence of the nucleotide sequence set out in any one of FIGS. 4 to 7; or
b. the amino acid sequence of a nucleotide sequence having at least 80%, preferably 90%, more preferably 95% homology, after optimal alignment, with the nucleotide sequence set out in any one of FIGS. 4 to 7; or
c. an amino acid sequence of a fragment of a papillomavirus protein as defined in a),
   in a process for the manufacture of a pharmaceutical composition for use in a prophylactic treatment against causative agents of lesions and carcinomas, more particularly cervical lesions and carcinomas.

According to the present invention the pharmaceutical composition will preferably be contained in a pharmaceutically acceptable medium, preferably a medium which can be injected into humans or given orally, which may, preferably, consist of water, or an aqueous saline solution, or of an aqueous solution based on dextrose and/or glycerol.

According to the present invention the pharmaceutical compositions as claimed may contain a pharmaceutically acceptable surfactant, such as for example anionic, cationic, non-ionic or amphoteric surfactants.

According to the invention the pharmaceutical compositions as claimed may contain pharmaceutically acceptable adjuvants and/or excipients which, preferably, may be administered by subcutaneous, intradermal or transmucousal route.

### BRIEF DESCRIPTION OF THE DRAWINGS

FIG. 1 is a plasmid map of pBGS1057;

FIG. 2 is a plasmid map of pART7;

FIG. 3 is a plasmid map of pART27;

FIG. 4 shows the nucleotide sequence SEQ ID NO 1, which is an isolated nucleotide sequence of a portion of the L1 gene;

FIG. 5 shows the nucleotide sequence SEQ ID NO 2, which is an isolated nucleotide sequence of a portion of the L1 gene;

FIG. 6 shows the nucleotide sequence SEQ ID NO 3, which is an isolated nucleotide sequence of a portion of the L1 gene;

FIG. 7 shows the nucleotide sequence SEQ ID NO 4, which is an isolated nucleotide sequence of a portion of the L1 gene;

FIG. 8 shows an electron micrograph of TMV-produced VLPs trapped onto an EM grid using HPV-16-specific MAb out of concentrated extracts from vector-infected plants;

FIG. 9 shows an electron micrograph of baculovirus-produced VLPs;

FIG. 10 shows an electron micrograph of a crude leaf extract from N. benthamiana infected with recombinant TMV (MAb V5 trapped VLPs in insert);

FIG. 11 shows an electron micrograph of a concentrated N. benthamiana extract trapped with MAb J4;

FIG. 12 shows an electron micrograph of a transgenic extract trapped with MAb J4;

FIG. 13 shows an electron micrograph of a transgenic extract trapped with MAb V5;

FIG. 14 shows a graph of the results of an analysis of sera from immunised rabbits. 1, 2 and 3 represent bleeds two weeks after immunisation; and

FIG. 15 shows a photograph depicting the results of rabbit sera tested by western blots with denatured VLPs produced in insect cells by recombinant baculovirus.

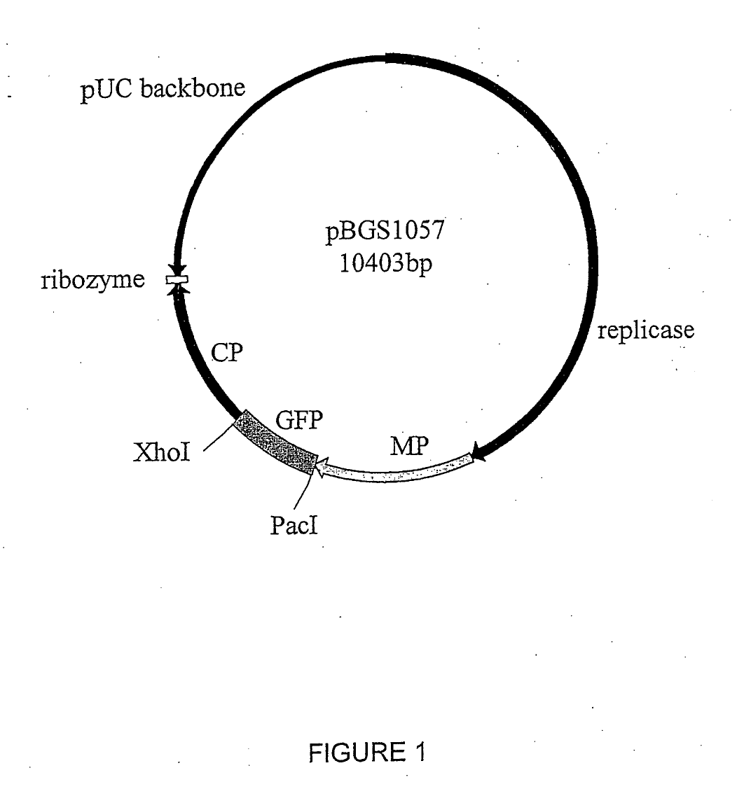

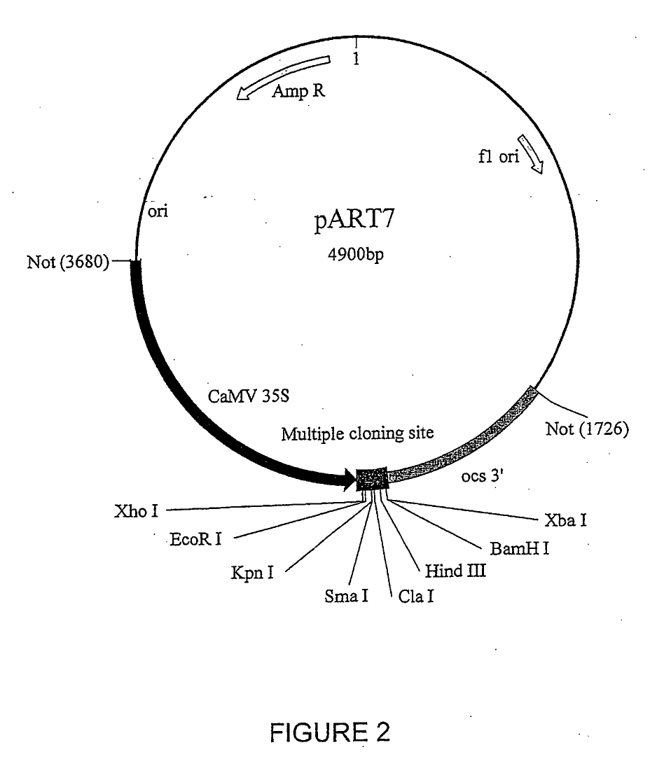

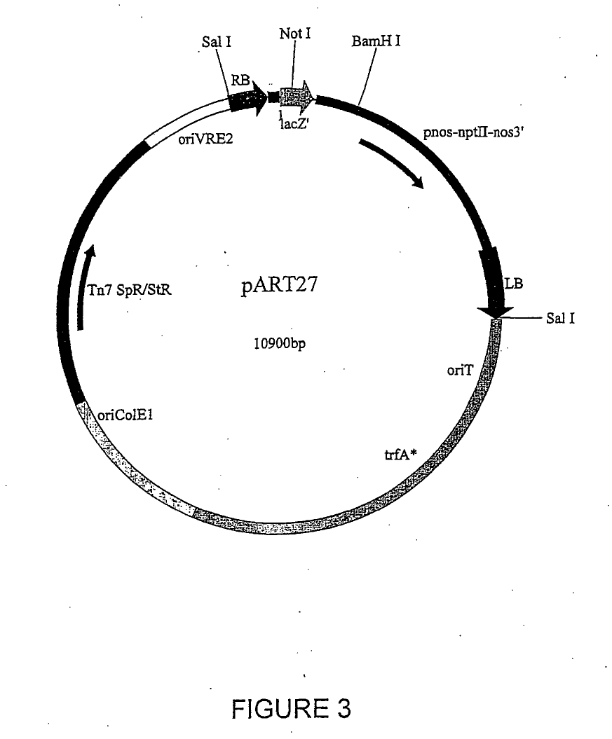

### DETAILED DESCRIPTION OF PREFERRED EMBODIMENTS OF THE INVENTION

The methods for preparing recombinant proteins and extracting said proteins are known to those skilled in the art and will not be developed in depth in the present description.

HPV 16 L1 Genes

Two different L1 genes were used for this study, SA-L1 and SAopt-L1. The amino acid (AA) sequence of these varied from the prototype sequence published by Seedoft et al. (1985) as outlined in Table 1.

TABLE 1
Amino acid differences of the variant HPV L1 genes

| | | AA differences compared to |
|---|---|---|
| Gene | Source | prototype sequence ( ) |
| SA-L1 | South African isolate | A (T) at 266 |
| SA-L1-NLS- | South African isolate | Deletion of AAs 484-504; stop at |
| | | 484 |
| SAopt-L1 | Optimised South | Identical to prototype |
| | African isolate | |
| SAopt-L1- | Optimised South | A (T) at 266; deletion of Ms 484- |
| NLS- | African isolate | 504; stop at 484 |

SA-L1 was optimised by site directed mutagenesis following the results published by Touze et al., 1998.

With the nuclear localisation signal (NLS) having been identified (Zhou et al., 1991), L1 genes were polymerase chain reaction (PCR) amplified without the nuclear localisation signal (last 21 AAs) for the L1 genes.

Cloning of the Genes into TMV pBSG 1057 Vector

All the L1 genes (including the NLS-) were cloned into pGEM-T easy plasmid with PacI and XhoI restriction sites (at 5′ and 3′ respectively) having been introduced during amplification by PCR. This was done to facilitate the subcloning of this gene fragment after sequencing into the pBSG 1057 vector (Large Scale Biology Corporation).

The green fluorescent protein (GFP) gene was excised from pBSG1057 by restriction enzyme digestion with PacI and XhoI, and L1 cloned into the TMV vector between these sites to produce the following constructs:
pBSG-O, pBSG-ON, pBSG-S, pBSG-SN,
R—Rochester isolate
S—South African isolate
O—Optimised South African isolate (Ala→Thr at AA 266)
N—Without nuclear localisation signal (NLS); ΔC483
   Transcription of pBSG Plasmids

mRNA of pBSG-O, pBSG-ON, pBSG-S, pBSG-SN, and pBSG1057 was transcribed using a Ribomax Transcription/translation kit (Promega). Ten micrograms of plasmid DNA was used for each reaction.

Infection of Nicotiana benthamiana with Recombinant TMV mRNA

Half of the mRNA (50 μl) was rubbed onto leaves of 3-week old N. benthamiana plants using cotton-wool buds and celite. Plants inoculated with the control pBSG1057 mRNA were monitored daily with a UV light for the appearance of GFP in both inoculated and upper leaves of the plants. Systemic spread of GFP was used as an indicator of systemic spread of recombinant TMV and leaves were sampled from all plants for detection of L1 protein on SDS polyacrylamide gels, for VLPs (“virus-like particles”) using EM and mRNA detection using RT-PCR.

Detection of L1 Protein

Crude protein preparations were made by crushing leaves in a mortar and pestle, or using Eppendorf pestles. Loading buffer was added samples and boiled for 10 minutes and run on 10% SDS polyacrylamide gels to separate the proteins.

Western blots were also performed on protein transferred onto nitrocellulose membranes from the SDS polyacrylamide gels. Membranes were probed with a mouse D9 monoclonal antibody (ex Christensen) at a dilution of 1:500.

Enzyme Linked Immunosorbent Assay (ELISA)

The ELISA plates were coated overnight with the plant derived antigen and washed and blocked with 1% non-fat milk before being probed with anti-VLP monoclonal antibody, V5 for 2 hrs. Following further washing the wells were probed with secondary anti-mouse (alkaline phosphatase labelled) antibody at 37° C. for an hour. The binding reactions were detected by calorimetric methods using NBT and BCIP at an optical density of 492 nm.

Bulk Purification of VLPs from N. benthamiana Infected with Various Recombinant TMV mRNA

The plant leaf material was homogenised in 1:2 volumes (plant material:buffer, w/v) of buffer (for HPV use PBS with 0.5M NaCl) and pressed out through cheesecloth over funnel until pulp dry. The extract was centrifuged for 10 mins at 4° C. (3000 rpm). 10% (w/v) PEG (MW 8000) was added to the supernatant and stirred overnight at 4° C. This was then centrifuged at 3000 rpm for 10 mins at 4° C. The pellet was resuspended in 1/10th the original volume in PBS (0.5M NaCl) overnight at 4° C. The mixture was centrifuged at 3000 rpm for 15 mins to remove denatured non-soluble components. The extract was overlaid onto a 40% sucrose cushion (made in PBS with 0.5M NaCl) and centrifuged at 100 000×g (for SW28, 24 000 rpm) at 10° C. for 2½ hrs. The pellet was resuspended in PBS-CsCl (0.4 g/ml CsCl) and the suspension was passed through 18 & 26 gauge needles to reduce viscosity. This was spun over night at 10° C. at 100 000×g. The TMV band was identified and the rest of material above was extracted and dialysed against PBS (0.5M NaCl). The dialysed material was spun down at 100 000×g onto a 40% sucrose cushion and resuspended in a small quantity of PBS (0.5M NaCl) and mixed thoroughly. This suspension was loaded onto a linear sucrose gradient of 20-60% and spun at 100 000×g (for SW 28, 24 000 rpm) at 10° C. for 3 hrs. The VLP band observed under scattered light was removed with a needle and syringe and dialysed against PBS (0.5M NaCl) at 4° C. for 12-24 hrs.

Electron Microscope Analysis

The VLPs were trapped onto carbon-coated copper grids coated with monoclonal antibody (conformation specific for HPV 16 L1) V5 and negatively stained with 2% uranyl acetate. The grids were viewed under the 200CX electron microscope.

Nicotiana benthamiana were infected with the RNA transcripts for each of 5 constructs. The plants infected with pBSG1057 was observed daily using a long wavelength UV light to detect the spread of the green fluorescent protein (GFP) from the site of inoculation. In general green spots were observed three days post inoculation on the inoculated leaves. The GFP was spread systemically on average 8 days post inoculation. The main symptom observed were the slight mosaic pattern on the leaves and the extensive curling of the leaves.

Infected leaf material was used to infect more plants by inoculating leaves with the crushed infected leaf material in water or phosphate buffer.

VLPs were isolated from plants that were infected with TMV constructs expressing the 4 different L1 genes. The VLPs were seen to bind the monoclonal neutralising antibody V5 by ELISA.

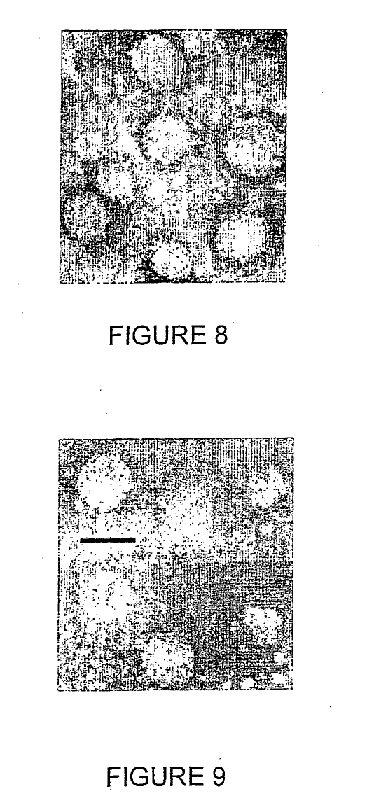

The VLPs observed under the electron microscope (trapped or untrapped with antibody) were similar in appearance to those as a result of the same gene expressed in an insect cell system (using baculoviruses), as shown in FIGS. 8 and 9, respectively (size bar indicates 70 nm). The size of the particles was observed to be approximately 50-60 nm in diameter and in crude preparations TMV particles were also observed. In one case (pBSG-SN infected) the necrosis was observed indicating that the L1 gene had been knocked out by TMV.

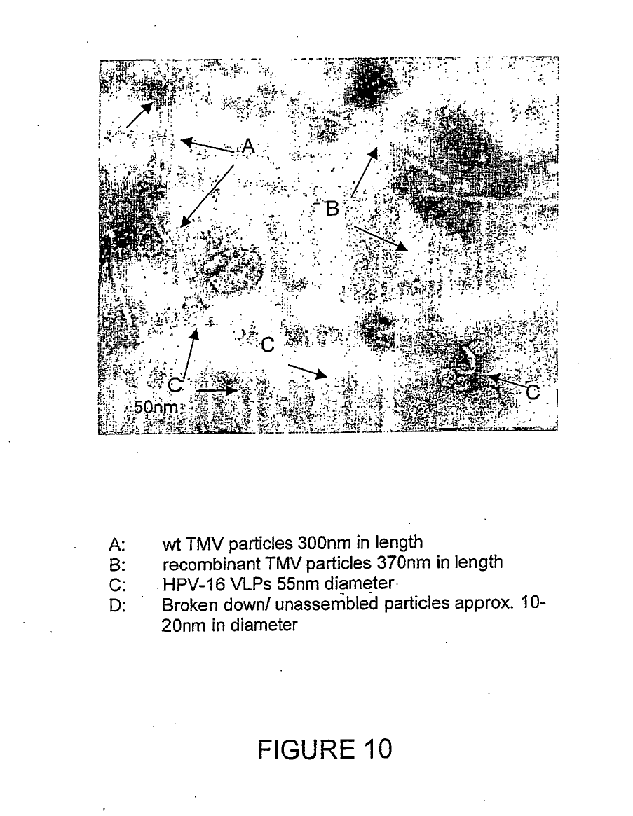

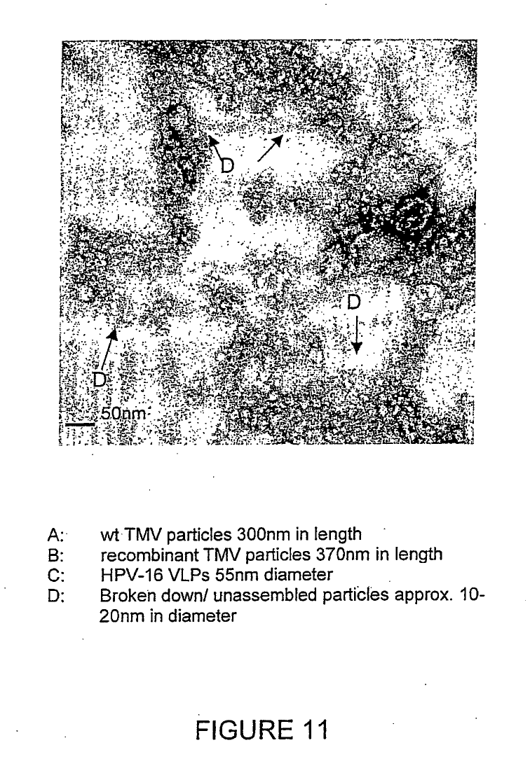

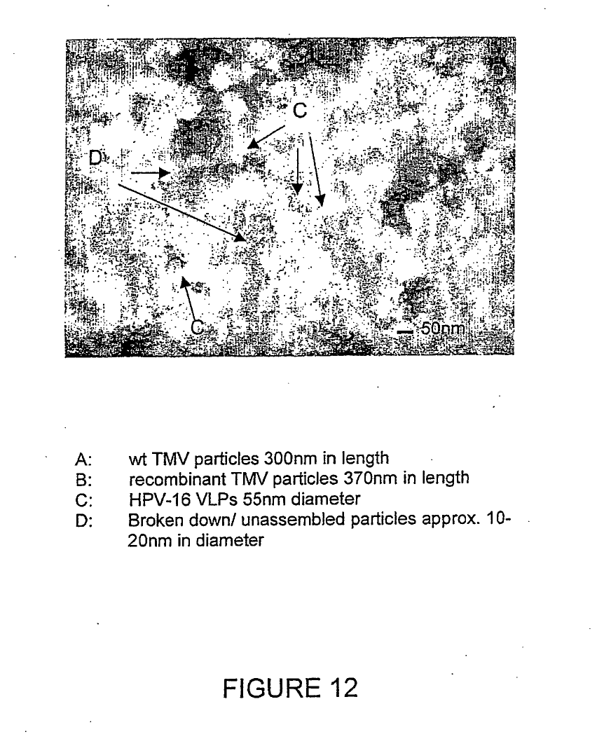

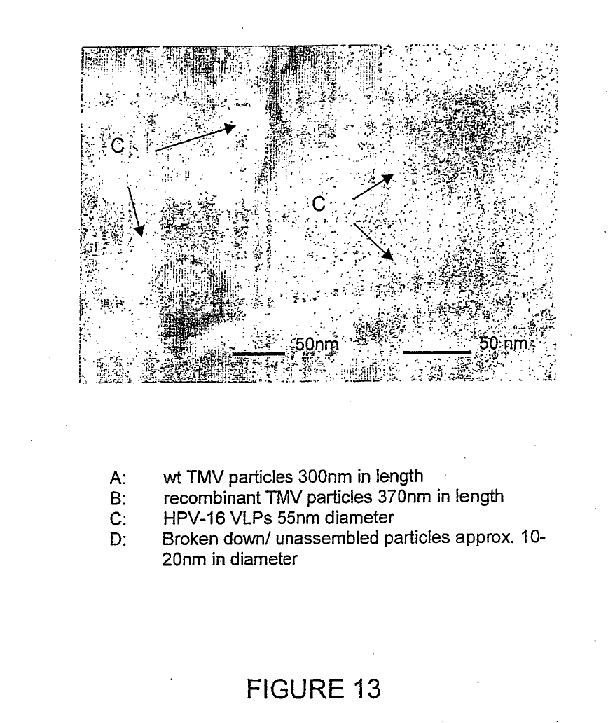

Referring to FIGS. 10 to 13, a series of micrographs confirm the production of VLPs from plants. The purified plant protein extract was viewed with a JEOL 200CX transmission electron microscope by immunotrapping the particles with J4 and V5 monoclonal antibodies at a dilution of 1/50. J4 binds a linear epitope in region aa 261-280 and V5 binds a conformation-specific epitope.

Immunisation of Rabbits with Plant Produced VLPs

Rabbits were immunised subcutaneous (2 sites) and intramuscular (1 site) with the plant produced VLPs and given a total of three boosters. VLP extracts were concentrated approximately 20 fold from initial concentrations.

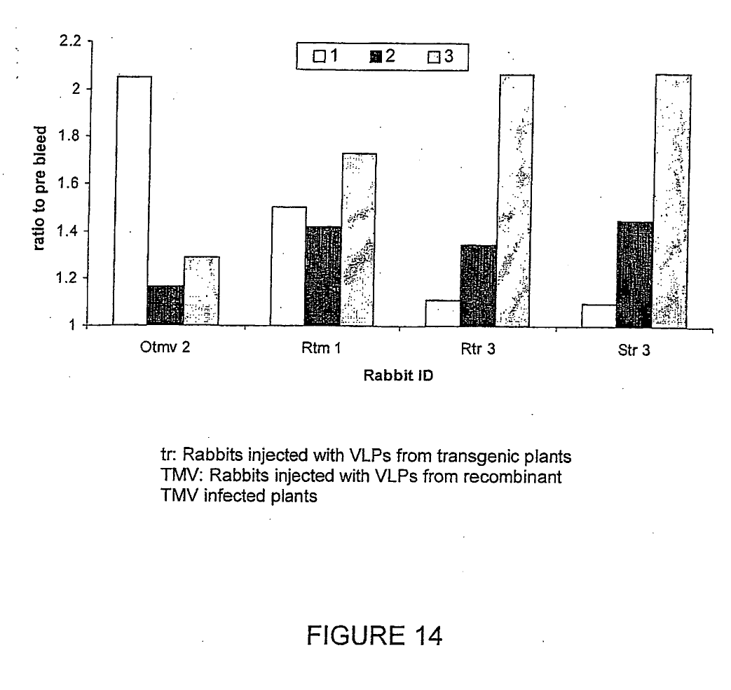

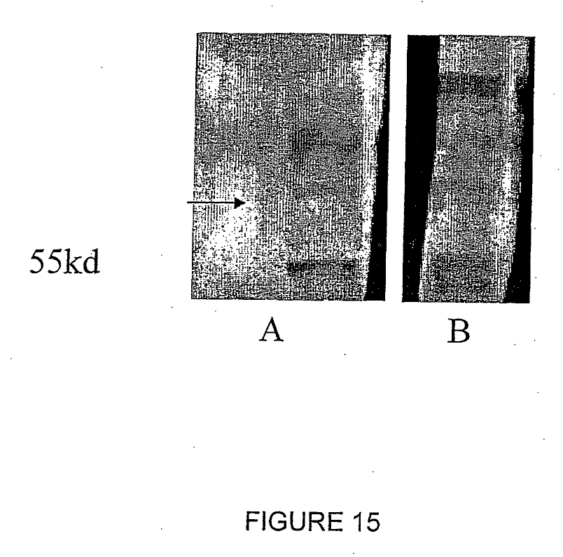

The rabbit serum was analysed for VLP specific antibodies by ELISA against baculovirus-produced HPV-16 L1 VLPs (FIG. 14) and by western blots (FIG. 15).

The crudely-purified HPV-16 VLPs were made from both TMV vector-infected and transgenic plants. The antisera produced reacted with baculovirus-produced VLPs, indicating that plant-produced VLPs are “antigenically appropriate”.

The effects of various N- and C-terminal deletions, as well as of a mutation abolishing assembly into VLPs, on the structural and assembly properties of baculovirus-produced HPV-16 L1 protein were investigated. Abolishing the C at aa 428 in L1 results in pentamers which cannot assemble into VLPs, which are highly stable. Deletion of 10 N-terminal, or up to 21 C-terminal amino acids, resulted in pentamers that still bound neutralisation-blocking V5 MAbs. This is an indication that sub-VLP-sized particles may be very useful vaccines for HPV as they may be easier to purify.

A number of different constructs of HPV-16 L1 protein and L1 protein lacking a nuclear localisation signal (NLS-, Δ483) have been expressed in plants via Tobacco mosaic virus vector infection, and it has been determined that these make T=7 icosahedral particle VLPs, are immunogenic in rabbits upon injection, and appear morphologically identical to VLPs made in a baculovirus expression system in insect cells. Additionally, it has been determined that HPV-16 VLPs made in infected plants bind a monoclonal antibody (MAb V5) which is conformationally specific and blocks neutralising antibody binding. These findings indicate that VLPs made in plants are functionally identical to VLPs made in insect cells. Further, the following constructs have been expressed via recombinant baculoviruses in insect cells, which bind the same conformationally-specific and sequence-specific MAbs as are bound by unaltered L1 protein:
a. HPV 16 L1 gene lacking the 10 N-terminal codons (ΔN10), which assembles into T=1 and also T=7 icosahedral particles;
b. HPV 16 L1 gene lacking 10 N-terminal codons and NLS (ΔN10Δ483), which assembles into T=1 and also T=7 icosahedral particles;
c. HPV 16 L1 gene with a C to G mutation (pen) at aa428 (pen504), which assembles into pentamers;
d. HPV 16 L1 gene with pen and ΔN10 modifications, which assembles into pentamers;
e. HPV 16 L1 gene with pen and Δ483 modifications, which assembles into pentamers; and
f. HPV 16 L1 gene with pen and ΔN10Δ483 modifications, which assembles into pentamers.

It is apparent to one skilled in the art that these constructs could be expressed in TMV vector infected plants and should behave in exactly the same way.

It will be immediately apparent to a person skilled in the art that although certain embodiments only of the invention have been set out herein, other modifications and/or variations of the invention are possible. Such modifications and/or variations are to be considered as falling within the scope of the present invention.

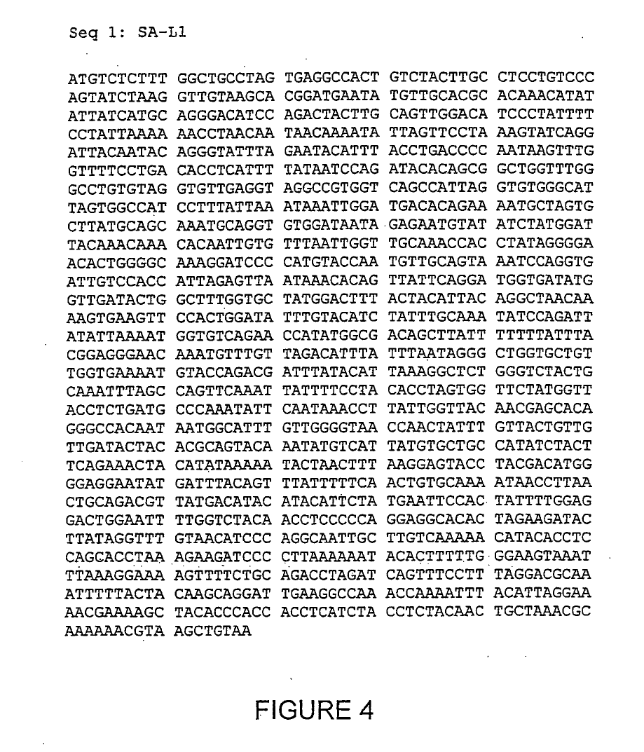

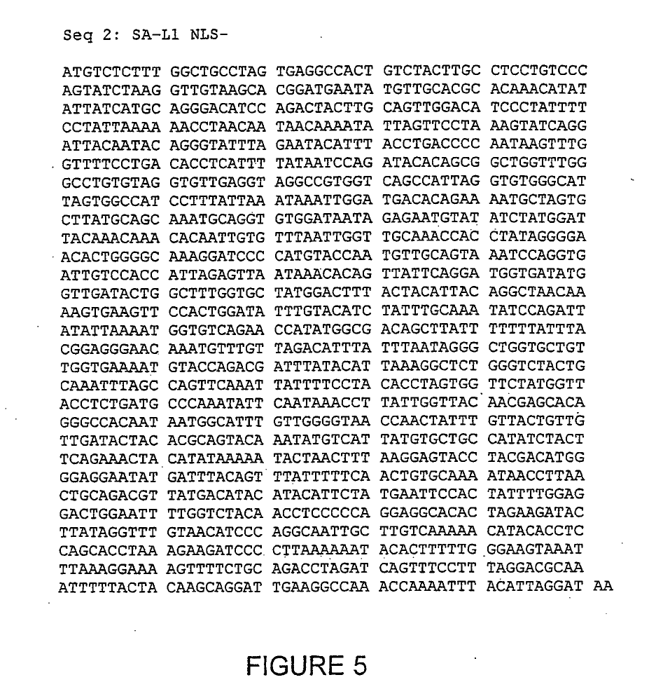

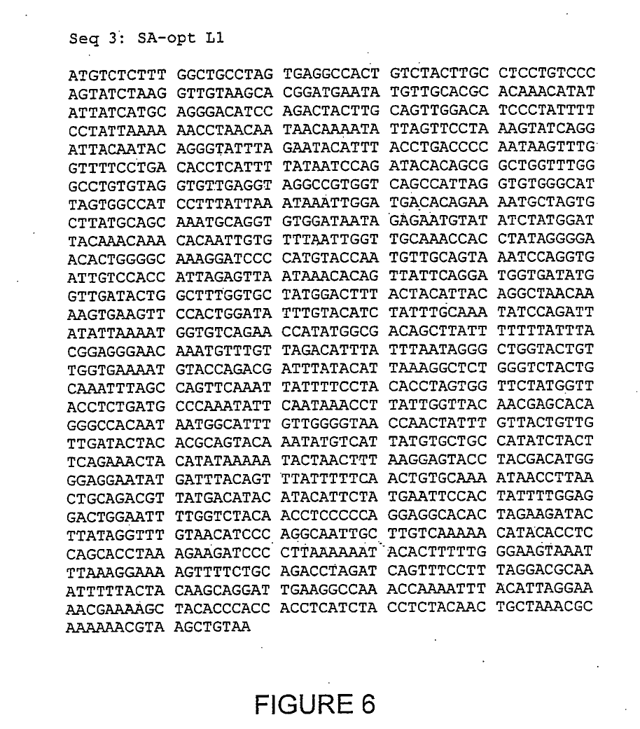

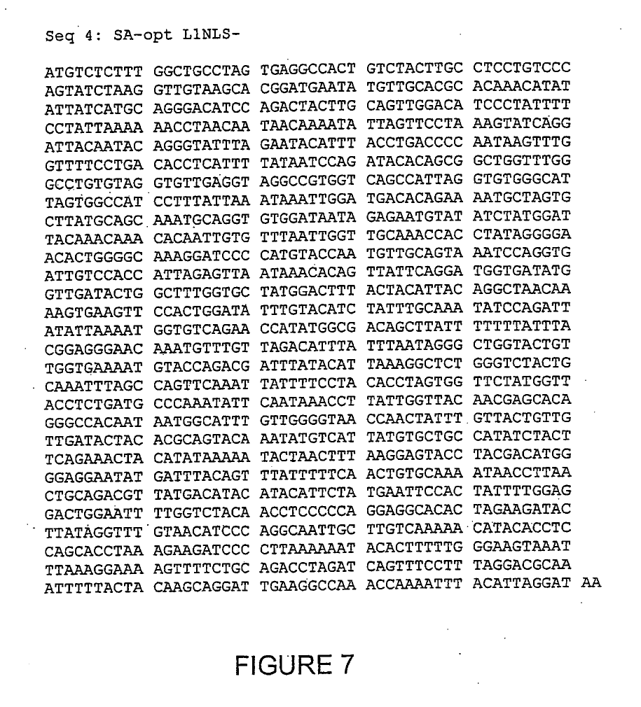

1. An isolated nucleotide sequence set out in any one of FIGS. 4 to 7, or part thereof.

2. A nucleotide sequence which is:
(i) a homologue, variant, and/or derivative of the nucleotide sequence of claim 1;
(ii) a complement of the nucleotide sequence of claim 1; or
(iii) a complement of the homologue, variant or derivative of (i).

3. A gene construct comprising a nucleotide sequence according to claim 1, and a promoter.

4. A vector comprising and/or expressing:
(a) an isolated nucleotide sequence set out in any one of FIGS. 4 to 7, or part thereof;
(b) a homologue, variant or derivative sequence set out in any one of FIGS. 4 to 7, or part thereof;
(c) a complement of the isolated nucleotide sequence set out in any one of FIGS. 4 to 7, or part thereof;
(d) a complement of the homologue, variant or derivative of the isolated nucleotide sequence set out in any one of FIGS. 4 to 7, or part thereof; or
(e) a nucleotide sequence that is capable of hybridizing to:
(1) the isolated nucleotide sequences, or parts thereof; or
(ii) the homologues, variants or derivatives of the isolated nucleotide sequences, or parts thereof; or
(iii) the complements of the isolated nucleotide sequences, or parts thereof; or
(iv) the complements of the homologues, variants or derivatives of the isolated nucleotide sequences, or parts thereof; or
(v) any combination of (e)(i)-(e)(iv); or
(f) any combination of (a)-(e),
and a promoter, wherein the promoter is operably linked to the sequence of any one of (a)-(f) or any combination of (a)-(f).

5. A method of producing a papillomavirus protein, or parts thereof, comprising the steps of:
1) contacting a plant cell with a gene construct of claim 3;
2) cultivating the plant cell under conditions suitable for the production of a protein or fragment of a protein of:
(a) the isolated nucleotide sequences of any one of FIGS. 4 to 7, or parts thereof; or
(b) homologues, variants or derivatives of the isolated nucleotide sequences, or parts thereof; or
(c) complements of the isolated nucleotide sequences, or parts thereof; or
(d) complements of the homologues, variants or derivatives of the isolated nucleotide sequences, or parts thereof; or
(e) a nucleotide sequence that is capable of hybridizing to:
i) the isolated nucleotide sequences, or parts thereof; or
ii) the homologues, variants or derivatives of the isolated nucleotide sequences, or parts thereof; or
iii) the complements of the isolated nucleotide sequences, or parts thereof; or
iv) the complements of the homologues, variants or derivatives of the isolated nucleotide sequences, or parts thereof; or
v) any combination of (e)(i)-(e)(iv);
(f) any combination of (a)-(e).

6. A method according to claim 5, wherein the plant cell is a Nicotiana benthamiana plant cell.

7. A method according to claim 5, wherein the conditions in step 2) permit the spontaneous assembly of the proteins or fragments of the proteins into immunogenic-response eliciting virus-like particles.

8. The use of a papillomavirus protein, or fragment of a papillomavirus protein, which comprises:
a) the amino acid sequence of the nucleotide sequence set out in any one of FIGS. 4 to 7; or
b) the amino acid sequence of a nucleotide sequence having at least 80% homology, after optimal alignment, with the nucleotide sequence set out in any one of FIGS. 4 to 7; or
c) an amino acid sequence of a fragment of a papillomavirus protein as defined in a),
in the manufacture of a pharmaceutical composition for use in a prophylactic treatment against causative agents of lesions and carcinomas.

9. The use of claim 8, wherein the pharmaceutical composition is used in a prophylactic treatment of cervical lesions and carcinomas.

10. A method of producing a papillomavirus protein, or parts thereof, comprising the steps of:
1) contacting a plant cell with a vector of claim 4;
2) cultivating the plant cell under conditions suitable for the production of a protein or fragment of a protein of:
(a) the isolated nucleotide sequences of any one of FIGS. 4 to 7, or parts thereof; or
(b) homologues, variants or derivatives of the isolated nucleotide sequences, or parts thereof; or
(c) complements of the isolated nucleotide sequences, or parts thereof; or
(d) complements of the homologues, variants or derivatives of the isolated nucleotide sequences, or parts thereof; or
(e) a nucleotide sequence that is capable of hybridizing to:
i) the isolated nucleotide sequences, or parts thereof; or
ii) the homologues, variants or derivatives of the isolated nucleotide sequences, or parts thereof; or
iii) the complements of the isolated nucleotide sequences, or parts thereof; or
iv) the complements of the homologues, variants or derivatives of the isolated nucleotide sequences, or parts thereof; or
v) any combination of (e)(i)-(e)(iv);
(f) any combination of (a)-(e).
